(19) United States
(12) Patent Application Publication
(10) Pub. No.: US 2013/0052635 A1
(43) Pub. Date: Feb. 28, 2013

(54) Method For Detecting Lipogenic Adenovirus

(71) Applicants: Richard L. Atkinson, Mechanicsville, MA (US); Jia He, Richmond, VA (US)

(72) Inventors: Richard L. Atkinson, Mechanicsville, MA (US); Jia He, Richmond, VA (US)

(21) Appl. No.: 13/580,009

(22) PCT Filed: Mar. 25, 2011
(86) PCT No.: PCT/US2011/029922

§ 371 Date: Oct. 30, 2012

Related U.S. Application Data
(60) Provisional application No. 61/317,526, filed on Mar. 25, 2010.

(51) Int. Cl. C12Q 1/70

(52) U.S. Cl. 435/5

(57) ABSTRACT

An immunoassay methods, immunoassay kits, and immunoassay devices for detecting lipogenic adenovirus infection in a subject. The immunoassay method includes using an antibody capture polypeptide, an antibody detection polypeptide, and optionally a competitor polypeptide.

### CROSS-REFERENCE TO RELATED PATENT APPLICATIONS

The present application claims the benefit of priority to U.S. Provisional Patent Application No. 61/317,526 filed Mar. 25, 2010, which is incorporated by reference.

### BACKGROUND OF THE INVENTION

ELISA assays may be used to rapidly detect antibodies to human adenovirus Ad-36 in human serum. Serum neutralization (SN) and hemagglutination-inhibition assays are classical reference methods for typing adenovirus. However, they are either very time-consuming or are cumbersome to perform.

### BRIEF SUMMARY OF THE INVENTION

According to an aspect of the invention, a method of detecting lipogenic adenovirus infection in a sample may include (a) providing a solid support coated with a first antibody capture polypeptide comprising at least 100 amino acids of an adenovirus type-36 (Ad-36) fiber coat protein, wherein the polypeptide does not include SEQ ID NO: 1, (b) contacting the sample to the antibody capture polypeptide such that anti-antibody capture polypeptide antibodies, if present in the sample, bind to the antibody capture polypeptide to form an antibody capture polypeptide—anti-antibody capture polypeptide antibody complex, (c) contacting the complex, if present, with an antibody detecting polypeptide comprising the at least 100 amino acids of the Ad-36 fiber coat protein; and (d) detecting the presence, absence, or quantity of specific binding of the detection polypeptide to the complex, thereby detecting a lipogenic adenovirus infection in the sample.

In some aspects, the method further comprises the step of applying the first antibody capture polypeptide comprising at least 100 amino acids of an adenovirus type-36 (Ad-36) fiber coat protein, wherein the polypeptide does not include SEQ ID NO: 1 to the solid support for a period of time to allow the first antibody capture polypeptide to adhere to a surface of the solid support.

In some aspects, the antibody capture polypeptide and/or the antibody detecting polypeptide comprises or consists of SEQ ID NO:10.

In some aspects, the antibody capture polypeptide comprises a non-adenovirus heterologous amino acid sequence and step (c) further comprises contacting the sample with a competitor polypeptide comprising the heterologous amino acid sequence such that anti-competitor polypeptide antibodies, if present in the sample, bind to the competitor polypeptide.

In some aspects, the competitor protein is a maltose binding protein.

In some aspects, the solid support is linked to one or more further antibody capture polypeptide that comprises a fragment of an adenovirus hexon protein.

In some aspects, the fragment of an adenovirus hexon protein comprise or consists of an amino acid sequence selected from the group consisting of SEQ ID NO: 4, SEQ ID NO: 5, SEQ ID NO: 6, and SEQ ID NO: 7.

In some aspects, the solid support is linked to one or more further antibody capture polypeptide that consists of a peptide selected from SEQ ID NO: 4, SEQ ID NO: 5, SEQ ID NO: 6, and SEQ ID NO: 7.

In some aspects, the antibody capture polypeptide comprises about 100 to about 400 amino acids of an Ad-36 fiber coat protein.

In some aspects, the Ad-36 fiber coat protein is SEQ ID No. 8.

In some aspects, the antibody detecting polypeptide is conjugated to a label that is capable of producing a measurable signal.

In some aspects, the label is selected from the group consisting of horseradish peroxidase (HRP), 1125, alkaline phosphatase, fluorescein isothiocyanate (FITC), tetramethyl rhodamine isothiocyanate (TRITC), green fluorescent protein (GFP), allophycocyanin, phycocyanin, phycoerythrin, and phycoerythrocyanin.

In some aspects, the sample is selected from the group consisting of biological sample, blood, semen, saliva, tears, nasal secretions, serum, cerebral fluid, urine, milk, and plasma.

According to another aspect of the invention, an assay kit for detecting lipogenic adenovirus may include a first antibody capture polypeptide comprising at least 100 amino acids of an adenovirus type-36 (Ad-36) fiber coat protein, wherein the polypeptide does not include SEQ ID NO: 1; and an antibody detecting polypeptide comprising the at least 100 amino acids of the Ad-36 fiber coat protein.

In some aspects, the antibody capture polypeptide and/or the antibody detecting polypeptide comprises or consists of SEQ ID NO:10.

In some aspects, the first antibody capture polypeptide is linked to a solid support.

In some aspects, the antibody capture polypeptide comprises about 100 to about 400 amino acids of an Ad-36 fiber coat protein.

In some aspects, the Ad-36 fiber coat protein is SEQ ID NO. 8.

In some aspects, the antibody capture polypeptide comprises a non-adenovirus heterologous amino acid sequence and the kit further comprises a competitor polypeptide comprising the heterologous amino acid sequence.

In some aspects, the competitor polypeptide is a maltose binding protein.

In some aspects, the kit further comprises, linked to the solid support, one or more further antibody capture polypeptide that comprises a fragment of an adenovirus hexon protein.

In some aspects, the fragment of an adenovirus hexon protein comprise or consists of an amino acid sequence selected from the group consisting of SEQ ID NO: 4, SEQ ID NO: 5, SEQ ID NO: 6, and SEQ ID NO: 7.

In some aspects, the kit further comprises a second antibody capture polypeptide comprises or consists of an amino acid sequence selected from SEQ ID NO:4 SEQ ID NO:5, SEQ ID NO:6, and SEQ ID NO:7.

In some aspects, the kit further comprises a label substrate, a wash solution, a blocking solution, a stopping solution, and a solid support.

In some aspects, the antibody detecting polypeptide is conjugated to a label.

In some aspects, the label is selected from the group consisting of horseradish peroxidase (HRP), 1125, alkaline phosphatase, fluorescein isothiocyanate (FITC), tetramethyl rhodamine isothiocyanate (TRITC), green fluorescent protein (GFP), allophycocyanin, phycocyanin, phycoerythrin, and phycoerythrocyanin.

According to a further aspect of the invention, an assay device used for detecting lipogenic adenovirus infection may include a solid support linked to (or coated with) a first antibody capture polypeptide comprising at least 100 amino acids of an adenovirus type-36 (Ad-36) fiber coat protein, wherein the polypeptide does not include SEQ ID NO: 1.

In some aspects, the antibody capture polypeptide comprises or consists of SEQ ID NO:10.

In some aspects, the solid support is coated with a second antibody capture polypeptide that comprises a fragment of an adenovirus hexon protein.

In some aspects, the second antibody capture polypeptide comprises or consists of a polypeptide selected from SEQ ID NO: 2, SEQ ID NO: 4, SEQ ID NO: 5, SEQ ID NO: 6, and SEQ ID NO: 7.

In some aspects, the antibody capture polypeptide comprises about 100 to about 400 amino acids of an Ad-36 fiber coat protein.

In some aspects, the Ad-36 fiber coat protein is SEQ ID NO. 8.

In some aspects, the solid support may be selected from a microtiter plate, a bead, a rod, nitrocellulose, a dipstick, or petri dish.

In some aspects, the assay device further comprises an anti-antibody capture polypeptide linked to the first antibody capture polypeptide forming an anti-antibody capture polypeptide—first antibody capture polypeptide complex.

In some aspects, the assay device further comprises an antibody detecting polypeptide linked to the complex.

In some aspects, the antibody detecting polypeptide is conjugated to a label.

Additional features, advantages, and embodiments of the invention may be set forth or apparent from consideration of the following detailed description, and claims. Moreover, it is to be understood that both the foregoing summary of the invention and the following detailed description are exemplary and intended to provide further explanation without limiting the scope of the invention as claimed.

### BRIEF DESCRIPTION OF THE FIGURES

FIG. 1 shows the optimized ELISA assay parameters.

FIG. 2 depicts the comparative sensitivity of the ELISA assay and the serum neutralization assay.

### DEFINITIONS

The invention is not limited to the particular methodology, protocols, and reagents, etc., described herein, as these may vary as the skilled artisan will recognize. The terminology used herein is used for the purpose of describing particular embodiments only, and is not intended to limit the scope of the invention. As used herein and in the appended claims, the singular forms “a,” “an,” and “the” include the plural reference unless the context clearly dictates otherwise. Thus, for example, a reference to “a cell” is a reference to one or more cells and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law.

Accordingly, provided immediately below is a “Definition” section, where certain terms related to the invention are defined specifically for clarity, but all of the definitions are consistent with how a skilled artisan would understand these terms. Particular methods, devices, and materials are described, although any methods and materials similar or equivalent to those described herein can be used in the practice or testing of the invention. All references referred to herein are incorporated by reference herein in their entirety.

“Ad-2” is Adenovirus type 2.

“Ad-36” is Adenovirus type 36.

“PPAR” is peroxisome proliferator activated receptors.

“CEBP” is CCAAT-enhancer binding protein.

“FAS is fatty acid synthase.

“Lipogenic adenovirus,” as used herein, generally refers to adenoviruses that are capable of stimulating increase lipid production in cells, tissues, and/or organs by facilitating expression of lipogenic enzymes, which in turn produce excess fatty acids and promote fat storage. Moreover, the lipogenic adenoviruses of the invention may cause or exacerbate malignant changes in cells. The lipogenic adenoviruses of the invention include but are not limited to Ad-36. While not intending to be limited to a particular mechanism, in some embodiments, the molecular mechanism of lipogenic adenovirus infection is the stimulation of lipogenic enzymes that increase fat deposition in adipose tissues and cause differentiation of adult stem cells in adipose tissue to adipocytes. Specific lipogenic enzymes may be over-expressed or expressed in the cell, such as fatty acid synthase (FAS), glycerol-3-phosphodehydrogenase, lipoprotein lipase (LPL), SREBP-1, SCD1, CPT 1, PPAR-gamma, and L-type pyruvate kinase. These lipogenic enzymes may be responsible for the formation of excess fatty acids and promote fat storage within the cells of multiple organs.

A “biological sample,” as used herein, generally refers to a sample of tissue or fluid from a human or animal including, but not limited to plasma, serum, spinal fluid, lymph fluid, the external sections of the skin, respiratory, intestinal and genitourinary tracts, tears, nasal secretions, saliva, blood cells, tumors, organs, tissue and sample of in vitro cell culture constituents.

“Subject,” as used herein, refers to humans or non-human animals.

An “isolated” or “substantially pure,” nucleic acid (e.g., DNA, RNA, or a mixed polymer) for example, is one which is substantially separated from other cellular components which naturally accompany a native human or animal sequence or protein, e.g., ribosomes, polymerases, many other human or animal genome sequences and proteins. The term embraces a nucleic acid sequence or protein which has been removed from its naturally occurring environment, and includes recombinant or cloned DNA isolates and chemically synthesized analogs or analogs biologically synthesized.

The term “antibody,” as used herein generally refers to antibodies, digestion fragments, specified portions and variants thereof, including antibody mimetics or comprising portions of antibodies that mimic the structure and/or function of an antibody or specified fragment or portion thereof, including single chain antibodies and fragments thereof. The invention encompasses antibodies and antibody fragments capable of binding to a biological molecule (such as an antigen or receptor), such as the fiber coat protein or other protein of lipogenic adenoviruses, and specifically, Ad-36, or portions thereof.

The term “nucleic acid sequence,” as used herein generally includes an oligonucleotide, nucleotide, or polynucleotide, and fragments thereof. The term is not limited by length and is generic to linear polymers of polydeoxyribonucleotides (containing 2-deoxy-D-ribose), polyribonucleotides (containing D-ribose), and any other N-glycoside of a purine or pyrimidine base, or modified purine or pyrimidine bases. These terms include double- and single-stranded DNA, as well as double- and single-stranded RNA.

The term “fragment,” as used herein generally includes any portion of a peptide or nucleic acid sequence. Peptide fragments retain at least one structural or functional characteristic of the subject polypeptides. Nucleic acid sequence fragments are greater than about 60 nucleotides in length, and most preferably includes fragments that are at least about 100 nucleotides, at least about 1000 nucleotides, and at least about 10,000 nucleotides in length. Peptide fragments are greater than about 10 amino acids in length, more preferably includes fragments that have at least about 25 amino acids, and even more preferably have at least about 100 amino acids.

### DETAILED DESCRIPTION OF THE INVENTION

### Immunoassays

The invention relates generally to immunoassay methodologies, immunoassay devices, and immunoassay kits for detecting lipogenic adenovirus infection in a subject. The inventors have surprisingly discovered that Ad-36 can be specifically detected without undesired cross-reaction with other non-lipogenic adenoviruses by detection of antibodies that specifically bind to particular Ad-36 peptides described herein. The immunoassay generally involves contacting with a biological sample from a subject an antibody capture polypeptide (detailed more specifically below) that is linked to a solid support and subsequently detecting specific binding of an antibody from the sample to the antibody capture polypeptide with an antibody detecting polypeptide. and optionally, a competitor polypeptide. Specific binding of antibodies from the sample to the antibody capture polypeptide indicates the presence of anti-lipogenic adenovirus antibodies in the sample, and is thereby indicative of lipogenic adenovirus infection in the subject. The antibody capture polypeptide can be linked to the solid support covalently or non-covalently as is generally known in the immunoassay arts.

In a particular aspect, the immunoassay may be carried out as follows: (i) a solid support may be coated with an antibody capture polypeptide, (ii) the solid support may be washed and then blocked with a blocking buffer, (iii) the solid support may be washed and an antibody detection antibody polypeptide conjugated to a label, a competitor polypeptide, and a sample may be added, (iv) the solid support may be washed and the appropriate label substrate may be added; (v) a stopping agent may be added, and (vi) the solid support may be examined for any detectable signal. In a more particular aspect, the polypeptide sequences of the antibody capture polypeptide and the antibody detection polypeptide are substantially identical. In an alternative aspect, the solid support may be coated with a first antibody capture polypeptide and a second antibody capture polypeptide.

As discussed in the examples, the inventors have found that an exemplary antibody capture polypeptide is an adipogenic adenovirus fiber coat protein that lacks at least the first 70 amino-terminal amino acids of the fiber coat protein. An exemplary adipogenic adenovirus fiber protein is the Adenovirus type-36 (Ad-36) fiber coat protein of SEQ ID No. 8.

In some embodiments, the antibody capture polypeptide comprises or consists of at least 100 amino acids of an Ad-36 fiber coat polypeptide, where the polypeptide does not include the first 70 amino-terminal amino acids of the fiber coat protein (e.g., does not comprise SEQ ID NO:1). In an even more specific embodiment, the antibody capture polypeptide may be the polypeptide of SEQ ID NO: 10. In additional embodiments, the antibody capture polypeptide may comprise at least one of an C-terminal truncated Ad36 fiber protein (1˜291aa), an N-terminal truncated Ad36 fiber protein-A (70˜371aa), an N-terminal truncated Ad36 fiber protein-B (183-371aa), an N-terminal truncated Ad36 fiber protein-C (195˜232aa), and an N-terminal truncated Ad36 fiber protein-D (233˜371 aa).

In an alternate embodiment, the solid support may be coated with a second antibody capture polypeptide. In particular, the second antibody capture polypeptide may be an Ad-36 hexon protein of SEQ ID No. 9. More particularly, the second antibody capture polypeptide may be a fragment of the Ad-36 hexon protein having at least 10 amino acids, more specifically, having at least 20 amino acids, even more specifically, having at least 50 amino acids, and most specifically, having at least 100 amino acids of the Ad-36 hexon protein. In specific embodiments, the second antibody capture polypeptide may comprise or consist of, at least one polypeptide selected from SEQ ID No. 2, SEQ ID No. 4, SEQ ID No. 5, SEQ ID No. 6, and SEQ ID No. 7.

In a specific embodiment, the antibody detection polypeptide may comprise an Ad-36 fiber coat protein of SEQ ID No. 8. In a more specific embodiment, the antibody capture polypeptide may comprise at least 100 amino acids of an Ad-36 fiber coat polypeptide, where the polypeptide does not include SEQ ID NO: 1. In additional embodiments, the antibody detection polypeptide may comprise at least one of an C-terminal truncated Ad36 fiber protein (1-291aa), an N-terminal truncated Ad36 fiber protein-A (70-371aa), an N-terminal truncated Ad36 fiber protein-B (183-371aa), an N-terminal truncated Ad36 fiber protein-C (195-232aa), and an N-terminal truncated Ad36 fiber protein-D (233371 aa). In certain aspects, the same polypeptide sequence for both the antibody capture polypeptide and the antibody detection polypeptide may be utilized for the immunoassay.

In a further embodiment, the antibody detection polypeptide is conjugated to a label. The label is capable of generating a measurable signal when it is contacted with the appropriate substrate. In some embodiments, the antibody detection polypeptide is conjugated to a label selected from horseradish peroxidase (HRP), I¹²⁵, alkaline phosphatase, fluorescein isothiocyanate (FITC), tetramethyl rhodamine isothiocyanate (TRITC), green fluorescent protein (GFP), allophycocyanin, phycocyanin, phycoerythrin, and phycoerythrocyanin, for example. In some embodiments, the antibody detection polypeptide is not labeled, and can be detected, for example, with a further secondary antibody, that is optionally labeled.

In certain aspects, the antibody capture polypeptide and/or the antibody detection polypeptide, may be made using a Mal protein fusion and purification system in E. coli. This system, however, may generate a polypeptide, that in addition to the desired Ad-36 amino acid sequences, may also contain non-adenovirus heterologous amino acid sequences. In a particular aspect, the non-adenovirus heterologous amino acid sequence may be the maltose binding protein (MBP). The inventors have discovered that subjects, and in particular, human subjects, may have significant amounts of antibodies to maltose binding protein. The anti-maltose binding protein antibodies may affect the quality and accuracy of the immunoassay. Therefore, in order to eliminate the non-specific antibodies that may be present in the sample, a competitor polypeptide, such as a MBP polypeptide of SEQ ID NO:3, may be added to the immunoassay. By adding the competitor polypeptide, the non-specific antibodies in the sample are captured by the competitor polypeptide, before they can bind to the antibody capture polypeptides coated on the solid support. Accordingly, in certain aspects, the competitor polypeptide is added concurrently with the sample and the antibody detecting polypeptide(s).

### Immunoassay Kits

According to other embodiments, an assay kit for detecting lipogenic adenovirus may include a first antibody capture polypeptide comprising at least 100 amino acids of an adenovirus type-36 (Ad-36) fiber coat protein, wherein the polypeptide does not include SEQ ID NO: 1; and an antibody detecting polypeptide comprising the at least 100 amino acids of the Ad-36 fiber coat protein. The antibody detection polypeptide may be conjugated to a label. In one particular aspect, the kit may contain a antibody capture polypeptide, an antibody detection polypeptide conjugated to a label, and a competitor polypeptide. In an alternate embodiment, the kit may contain a first antibody capture polypeptide, a second antibody capture polypeptide, an antibody detection polypeptide conjugated to a label, and a competitor polypeptide. The kits of the invention may further contain at least one of following reagents for carrying out the immunoassay such as blocking buffer, stopping reagents, a label substrate, a solid support, and washing solutions, for example.

In a specific embodiment, the kit may include an antibody capture polypeptide of SEQ ID No. 10, a maltose binding protein of SEQ ID No. 3, and an antibody detection polypeptide of SEQ ID No. 10 conjugated to a label. The kit may also contain a second antibody capture polypeptide having a peptide sequence selected from a peptide of SEQ ID No. 2, SEQ ID NO: 4, SEQ ID NO: 5, SEQ ID NO: 6, or SEQ ID NO: 7. The kit may further contain a solid support, a blocking buffer, a washing solution, and a label substrate.

### Immunoassay Devices

According to other embodiments, an assay device used for detecting lipogenic adenovirus infection may include a solid support coated with a first antibody capture polypeptide comprising at least 100 amino acids of an adenovirus type-36 (Ad-36) fiber coat protein, wherein the polypeptide does not include SEQ ID NO: 1. In a more specific aspect, the antibody capture polypeptide may comprise a polypeptide of SEQ ID NO: 10. Further, the solid support may be coated with a second antibody capture polypeptide. The second antibody capture polypeptide may comprise at least 10 amino acids of the hexon polypeptide of SEQ ID No. 9. Specifically, the second antibody capture polypeptide may be a polypeptide selected from SEQ ID NO:2, SEQ ID NO: 4, SEQ ID NO: 5, SEQ ID NO: 6 or SEQ ID NO: 7.

In a further embodiment, the immunoassay device of the invention may include an anti-lipogenic adenovirus antibody linked to at least one of the first or second antibody capture polypeptides to form an anti-lipogenic adenovirus antibody—antibody capture polypeptide complex. Further, the immunoassay device may also include an antibody detecting polypeptide having a label linked to the complex. The antibody detecting polypeptide and the antibody capture polypeptide may have substantially identical amino acid sequences. In a specific aspect, the antibody capture polypeptide and the antibody detecting polypeptide may be a peptide selected from at least 100 amino acids of an adenovirus type-36 (Ad-36) fiber coat protein, wherein the polypeptide does not include SEQ ID NO: 1, SEQ ID No. 10, a C-terminal truncated Ad36 fiber protein (1-291aa), an N-terminal truncated Ad36 fiber protein-A (70-371aa), an N-terminal truncated Ad36 fiber protein-B (183-371aa), an N-terminal truncated Ad36 fiber protein-C (195-232aa), or an N-terminal truncated Ad36 fiber protein-D (233-371aa).

In a specific embodiment, the solid support may be a microtiter plate, dipstick, a bead, nitrocellulose, a rod, or a petri dish.

Without further elaboration, it is believed that one skilled in the art using the preceding description can utilize the invention to the fullest extent. The following examples are illustrative only, and not limiting of the disclosure in any way whatsoever.

### EXAMPLES

### Specific Example 1

TABLE 1

| | | | |
|---|---|---|---|
| Approaches | Condition | Results | Conclusion |

Tested four Ad36 specific peptides:

| | | | |
|---|---|---|---|
| WGKVYKDPIPYD (12 aa) | Traditional | Cannot be recognized | Not applicable |
| | sandwich | by positive control | |
| | ELISA¹ | serum | |
| VETARDSKLT(10 aa) | Traditional | Cannot be recognized | Not applicable |
| | sandwich | by positive control | |
| | ELISA¹ | serum | |
| GTGSSAHG(8 aa) | Traditional | Cannot be recognized | Not applicable |
| | sandwich | by positive control | |
| | ELISA¹ | serum | |
| LGGEVYQPGFIVVK (14 aa) | Traditional | Cannot be recognized | Not applicable |
| | sandwich | by positive control | |
| | ELISA¹ | serum | |

Tested seven various recombinant viral proteins (purified with pMal
expression system and carry an in-fusion protein: Maltose Binding
Protein) in sandwich ELISA:

| | | | |
|---|---|---|---|
| Full length Ad36 | Traditional | Can be recognized by | Not specific |
| fiber | sandwich | positive control serum, | |
| protein (371 aa) | ELISA¹ | but cannot distinguish | |
| | | SN positive and | |
| | | negative human serum | |
| C-terminal truncated | Traditional | Can be recognized by | Not specific |
| Ad36 fiber protein | sandwich | positive control serum, | |
| (1~291 aa) | ELISA¹ | but cannot distinguish | |
| | | SN positive and | |
| | | negative human serum | |
| N-terminal truncated | Traditional | Can be recognized by | Not specific |
| Ad36 fiber protein- | sandwich | positive control serum, | |
| A (71~371 aa) | ELISA¹ | but cannot distinguish | |
| | | SN positive and | |
| | | negative human serum | |
| N-terminal truncated | Traditional | Can be recognized by | Not specific |
| Ad36 fiber protein- | sandwich | positive control serum, | |
| B (183~371 aa) | ELISA¹ | but cannot distinguish | |
| | | SN positive and | |
| | | negative human serum | |
| N-terminal truncated | Traditional | The protein was not | Not available |
| Ad36 fiber protein- | sandwich | available | |
| C (195~232 aa) | ELISA¹ | | |
| N-terminal truncated | Traditional | The protein was not | Not available |
| Ad36 fiber protein- | sandwich | available | |
| D (233~371 aa) | ELISA¹ | | |
| Truncated Ad36 hexon | Traditional | Cannot be recognized | Not applicable |
| protein-E | sandwich | by positive control | |
| (142~450 aa) | ELISA¹ | serum | |

Tested N-terminal truncated Ad36 fiber protein-A in double
antigen ELISA system:

| | | | |
|---|---|---|---|
| N-terminal truncated | Double-antigen | Can be recognized by | Not specific enough |
| Ad36 fiber protein- | ELISA system² | positive control serum | |
| A (71~371 aa) | | and can detect some | |
| | | SN positive serum | |

Purified recombinant Ad36 and Ad2 fiber protein and tested
in competitive double-antigen ELISA system:

| | | | |
|---|---|---|---|
| N-terminal truncated | Competitive | Cannot be recognized | Not applicable |
| Ad36 fiber protein | double-antigen | by positive control | |
| (71~371 aa) and Ad2 | ELISA system3 | serum | |
| fiber protein | | | |
| (69~578 aa) with | | | |
| His-tag | | | |

Purified Ad-36 native hexon protein from live virus and tested
in double-antigen ELISA system:

| | | | |
|---|---|---|---|
| Ad-36 whole hexon | Double-antigen | Can be recognized by | Not specific |
| protein (Trimer) | ELISA system² | positive control serum, | |
| | | but cannot distinguish | |
| | | SN positive and | |
| | | negative human serum | |

Tested recombinant Ad36 fiber protein and E. coli Maltose
Binding Protein (MBP) in competitive double-antigen
ELISA system:

| | | | |
|---|---|---|---|
| N-terminal truncated | Competitive | Can be recognized by | Applicable but |
| Ad36 fiber protein- | double-antigen | positive control serum | may be improved |
| A (71~371 aa) and | ELISA system³ | and can detect some | by hexon peptides |
| E. coli Maltose | | SN positive serum | |
| Binding Protein | | with false negative | |
| (MBP) | | rate of ~5% and ~15% | |
| | | false positive rate. The | |
| | | background was high | |

Optimized ELISA condition and identified five peptides
from Ad-36 hexon protein that may increase our assay
sensitivity:

| | | | |
|---|---|---|---|
| N-terminal truncated | Improved | Significantly reduced | Applicable but needs |
| Ad36 fiber protein- | competitive | background and false | more improvement |
| A (71~371 aa) and | double-antigen | positive rate. | |
| E. coli Maltose | ELISA system⁴ | | |
| Binding Protein | | | |
| (MBP) | | | |
| TDKERQNGGQPPTTKDVT | Improved | | Potential optimizer |
| (18 aa) | competitive | | peptide |
| (SEQ ID No. 7) | double-antigen | | |
| | ELISA system⁴ | | |
| DKGLQIGEDENNEDGEEEIYADK | Improved | | Potential optimizer |
| (23 aa) | competitive | | peptide |
| (SEQ ID No. 2) | double-antigen | | |
| | ELISA system⁴ | | |
| AKFLNGENGQPSKDQDIT | Improved | | Potential optimizer |
| (18 aa) | competitive | | peptide |
| (SEQ ID No. 4) | double-antigen | | |
| | ELISA system⁴ | | |
| DLKQNDTGTTQNQPD | Improved | | Potential optimizer |
| (15 aa) | competitive | | peptide |
| (SEQ ID No. 5) | double-antigen | | |
| | ELISA system⁴ | | |
| KYENGAGNGSWKVDGE | Improved | | Potential optimizer |
| (16 aa) | competitive | | peptide |
| (SEQ ID No. 6) | double-antigen | | |
| | ELISA system⁴ | | |

Traditional sandwich ELISA¹: Step 1. Coat microplate with coating protein → Step 2. Wash plate and then block plate with blocking buffer → Step 3. Wash plate and then add diluted serum → Step 4. Wash plate and then add HRP labeled secondary antibody (anti human IgG or anti human IgM) → Step 5. Wash plate and add HRP subtract → Step 6. Add stop agent
Double-antigen ELISA system²: Step 1. Coat microplate with coating antigen → Step 2. Wash plate and then block plate with blocking buffer → Step 3. Wash plate and then add serum → Step 4. Wash plate and then add HRP conjugated antigen (the same antigen as the coating antigen) → Step 5. Wash plate and add HRP subtract → Step 6. Add stop agent
Competitive double-antigen ELISA system³: Step 1. Coat microplate with coating antigen → Step 2. Wash plate and then block plate with blocking buffer → Step 3. Wash plate and then add serum → Step 4. Wash plate and then add HRP conjugated antigen (the same antigen as the coating antigen) plus competitor protein → Step 5. Wash plate and add HRP subtract → Step 6. Add stop agent
Improved competitive double-antigen ELISA system⁴: Step 1. Coat microplate with coating antigen → Step 2. Wash plate and then block plate with blocking buffer → Step 3. Wash plate and then add HRP conjugated antigen (the same antigen as the coating antigen) plus competitor protein, and serum → Step 4. Wash plate and add HRP subtract → Step 5. Add stop agent

### Specific Example 2

### Tested Four Ad36 Specific Peptides in Traditional Sandwich ELISA

The fiber protein sequence was submitted to a program that could use the method described by Kolaskar and Tongaonkar (1990) to predict the antigenic peptides on submitted protein sequence. 14 peptide sequences were identified that have highest antigenic propensity. Four peptides that are unique to Ad-36 and located on the C-terminus were selected, synthesized and tested in traditional sandwich ELISA.

Results:

All four peptides cannot be recognized by positive control serum, suggesting that they may not possess antigen epitopes.

### Specific Example 3

### Tested Seven Various Recombinant Viral Proteins in Traditional Sandwich ELISA

Seven recombinant viral proteins using PMAL protein fusion and purification system from New England BioLabs were constructed. This system has been used to express various virus surface antigens in E. coli (Rhyum S B et al., 1994) and is uncommonly effective at promoting the solubility of polypeptides to which it is fused. In addition, the purification procedure is very cost-efficient and very easy to manipulate. Therefore this system was chosen to first to express our recombinant proteins in E. coli. However, the recombinant proteins generated by this expression system carry a 42 kD fusion protein: maltose binding protein as a purification tag.

Results:

All seven recombinant proteins from E. coli except N-terminal truncated Ad36 fiber protein-C and D as listed in the Table 1, above, were purified and obtained. Purified proteins were coated on 96 well high binding capacity microplates and tested in a traditional ELISA assay system involving secondary antibodies. The positive control rabbit serum could recognize four recombinant proteins except truncated Ad36 hexon protein-E. However, both positive and negative human serum that had been tested in serum neutralization assay could react with those four proteins to a similar degree. There is poor correlation between ELISA results and serum neutralization assay results. Thus, the ELISA appeared sensitive, but not specific.

### Specific Example 4

### Tested N-Terminal Truncated Ad36 Fiber Protein-A in Double Antigen ELISA System

ELISA plates with high-binding capacity were coated with antigens overnight and then washed several times before blocking 2% BSA in PBS were used for blocking at room temperature for 1 hour. After several washes, test sera were added into the plate and then incubated at room temperature for 1 hour. The same antigens that have been labeled with HRP (Horseradish peroxidase) were then added into the plates. However for the traditional sandwich ELISA, HRP labeled secondary antibodies (anti human IgG, anti human IgM etc) were used instead. After incubation for 30 mins at room temperature, the plate was washed thoroughly. HRP subtracts were then added into plates. Blue color shows up after a few minutes. The reaction then can be stopped by 1N HCl and read at 450 nm wavelength.

The main differences between this double antigen ELISA system and traditional sandwich ELISA is that the double antigen ELISA does not use secondary antibodies, so it can detect both IgG and IgM antibodies.

Results:

This new method using our recombinant N-terminal truncated Ad36 fiber protein-A (70aa-371aa). It worked very well for the rabbit serum. The positive rabbit serum had more than 10 times higher readings than the negative rabbit serum.

### Specific Example 5

### Purified Recombinant Ad36 and Ad2 Fiber Protein and Tested in Competitive Double-Antigen ELISA System

The following proteins were expressed and purified: Ad-36 fiber protein (same construct as N-terminal truncated Ad36 fiber protein-A) and Ad-2 fiber protein (similar construct) in pET H is Tag expression system (Novagen). The purified protein carries a His Tag that is composed of six histidines.

Results:

These two constructs produced abundant proteins; however both proteins had to be purified under denaturing condition with 6M urea. The proteins were further dialyzed in PBS to remove the urea. A competitive double antigen ELISA was performed in which we used 100 times more of unlabeled Ad-2 fiber protein with HRP-conjugated Ad-36 fiber protein. The positive rabbit serum cannot be recognized in this system. It is possible that both recombinant proteins did not form an appropriate structural form that can be recognized by Ad-36 antibodies.

### Specific Example 6

### Purified Ad-36 Native Hexon Protein from Live Virus and Tested in Double-Antigen ELISA System

Both adenovirus fiber and hexon proteins form trimers. Many studies have showed that both anti-fiber and anti-hexon antibodies preferentially recognize their native trimeric form. It has also been showed that hexon possess both type specific and genus specific antigens. In addition some commercially available adenovirus ELISA kits used purified hexon protein (such as Serion classice adenovirus IgG/IgA ELISA). Therefore we purified major Ad-36 capsid protein: hexon from live virus infected cells and tested it in the double-antigen ELISA system.

Results:

We obtained adequate native Ad-36 hexon protein. It can be recognized by positive rabbit serum. However, it correlates poorly with Ad-36 serum neutralization results.

### Specific Example 7

### Tested Recombinant Ad36 Fiber Protein and E. coli Maltose Binding Protein (MBP) in Competitive Double-Antigen ELISA System

Human serum contains significant amount of antibodies to E. coli Maltose Binding Protein (MBP). Since the recombinant N-terminal truncated Ad36 fiber protein-A (70α-371aa) used in the approach 3 carries MBP fusion protein, the MBP antibodies in human serum may attribute to the non-specificity. Therefore, a competitive double-antigen ELISA was developed in which purified E. coli Maltose Binding Protein (MBP) was added into the system to remove non-specific antibodies.

We tested total 89 human serum samples using serum neutralization assay and our competitive ELISA in April 2009. 12 are positive, 76 are negative and one of them is equivocal according to the serum neutralization assay. ELISA assay showed that 17 samples are positive if we use 0.898 as a cutoff for positive and 72 are negative. However, there are 9 false positives and 3 false negatives whose ELISA results do not correlate with serum neutralization results. Therefore the false positive rate is 10.11% and false negative rate is 3.37%. Statistical analysis showed that ELISA results correlate with SN results.

106 samples were tested in addition to initial 89 human serum samples previously tested using competitive ELISA and SN assay side by side to see how well they correlate. According to SN assay, 14 are positive, 90 are negative and 2 are equivocal. ELISA assay showed that 36 samples were positive if 0.58 is used as a cutoff for positive, and 70 are negative. However, there are 26 false positives and 4 false negatives whose ELISA results do not correlate with serum neutralization results. Therefore the false positive rate is 24.5% and the false negative rate is 3.7%. The statistical analysis showed that ELISA results correlated highly with SN results (p-Value=0.0076, excluded two SN equivocal samples).

This ELISA assay correlates well with SN assay, however the background is high that might contribute to the high false positive rate.

### Specific Example 8

### Optimized ELISA Condition and Identified Five Peptides from Ad-36 Hexon Protein that May Increase Our Assay Sensitivity

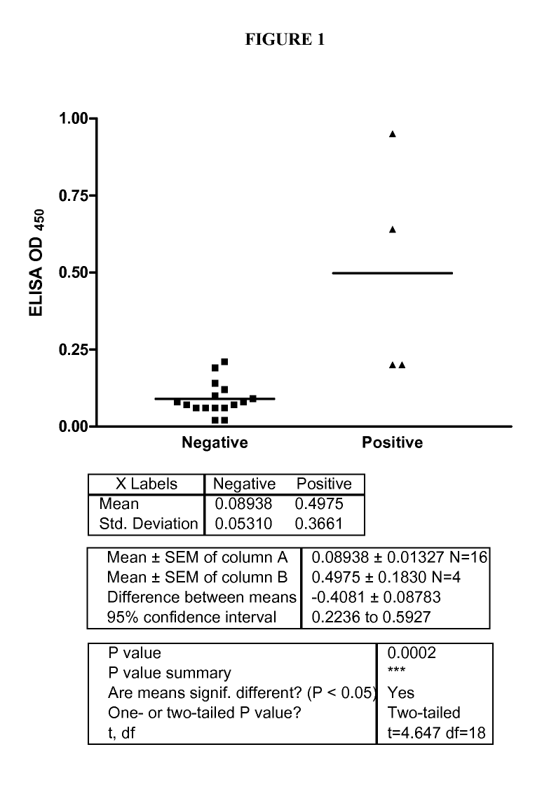

Different parameters and different combinations of the parameters including different dilution for coating antigen, HRP-labeled antigen and serum samples and different amount of MBP, and also different substrate-incubation time was used for optimization. A total 12 different conditions were tested, of which one condition worked the best. FIG. 1 shows the results after optimization. Both conditions can separate SN negative and SN positive samples.

There are several significant improvements after optimization. Firstly, the number of false positive samples dramatically decreased. Three samples were false positive before optimization (055A, 097A and 101A). Only one sample is false positive after optimization (097A). The number of false negative remains the same. Second, the average absorbance for SN negative samples using initial condition is 0.47. In contrast, it is only 0.09 using optimized condition, which is more than five-fold decrease and can barely be seen by eyes. Third, the difference in their absorbance value between SN negative and positive samples is greater after optimization. Using initial condition, SN positive samples have about three time higher absorbance than SN negative samples. However the differences were doubled after optimization and SN positive samples have six time higher absorbance than SN negative samples. Lastly, after optimization we combined two steps together, so we can save about 1.5 hour for one plate. In addition, we only need to use half of serum sample, half of HRP-labeled antigen and ¼ of MBP compared to initial condition, so it is more cost-efficient.

### Specific Example 9

### Comparison of Sensitivity of Serum Neutralization Assay Versus ELISA Assay on Same Samples

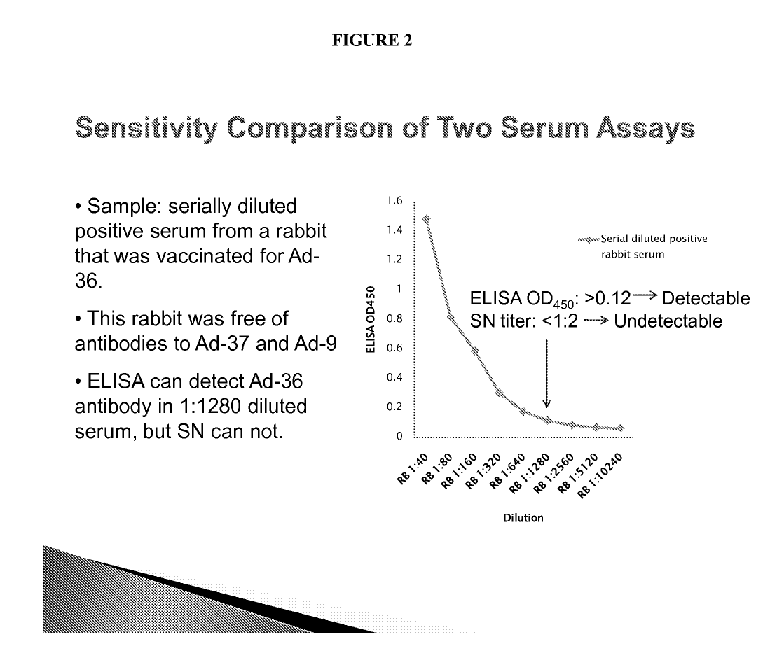

Serum neutralization has been used as the “gold standard” assay for detecting viruses. However, there are questions about the sensitivity and specificity of each assay versus the other. To determine the sensitivity of the serum neutralization assay versus the ELISA assay, positive serum from a rabbit that was vaccinated for Ad-36 was serially diluted from a titer of 1:40 to a titer of 1:10240. The rabbit was free of antibodies to Adv37 and Adv9, but had a titer of antibodies to Adv36 that at 17 serial dilutions was capable of neutralizing viral activity against A549 cells. As shown in FIG. 2 below, the ELISA was significantly more sensitive than the serum neutralization assay. ELISA could detect Ad-36 antibody two dilutions below the point at which serum neutralization could do so. This illustrates that the ELISA assay is more sensitive than serum neutralization and that samples that may be called “false positive” in the ELISA assay compared to the serum neutralization assay likely are indeed positive for Adv36 antibodies.

### Specific Example 10

### Comparison of Serum Neutralization Assay Versus ELISA Assay on Same Samples

Samples were obtained from Finland on a group of 252 subjects and were assayed by serum neutralization assay and ELISA at approximately the same time and at the same cycle of freeze-thaw. As can be seen in Table 2 below, the rate of Adv36 positive samples with serum neutralization assay was 13% and with ELISA was 36%. The ELISA had a 4% “equivocal” rate. There were 72 samples that were SN Negative, ELISA Positive and 7 samples that were SN Positive, ELISA negative. If serum neutralization is used as the “gold standard,” for the total number of samples there was a 29% “false positive” rate by ELISA assay and a 3% “false negative” rate. However, as noted above in Specific Example 9, the ELISA is more sensitive using a known positive sample for both assays. Therefore, the ELISA assay has a very low rate of “missing” positive samples, but a high rate of detecting samples that serum neutralization misses. This shows that the ELISA is both highly sensitive and highly specific.

TABLE 2
Comparison of Serum Neutralization
Assay and ELISA Assay in human samples
OVERALL SUMMARY OF SN AND ELISA ASSAYS

| | Number | Percent |
|---|---|---|
| Serum Neutralization Assay | | |
| SN Negative | 220 | 87% |
| SN Positive | 32 | 13% |
| Totals: | 252 | 100% |
| ELISA Assay | | |
| ELISA Negative | 152 | 60% |
| ELISA Positive | 91 | 36% |
| ELISA Equivocal | 9 | 4% |
| Totals: | 252 | 100% |
| “False Positive/False Negative” Rate | | |
| SN Negative, ELISA Positive | 72 | 29% |
| SN Positive, ELISA Negative | 7 | 3% |

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in cellular and molecular biology, chemistry, or in the relevant fields are intended to be within the scope of the appended claims.

The disclosures of all references and publications cited above are expressly incorporated by reference in their entireties to the same extent as if each were incorporated by reference individually.

SEQUENCE LISTING
SEQ ID NO: 1
MSKRLRVEDDFNPVYPYGYARNQNIPFLTPPFVSSDGFQNFPPGVLSLKL
ADPIAIANGNVSLKVGGGLT
SEQ ID No. 2
DKGLQIGEDENNEDGEEEIYADK
SEQ ID No. 3
(MBP protein sequence used in our double
antigen ELISA (412 aa)):
MKIKTGARILALSALTTMMFSASALAKIEEGKLVIWINGDKGYNGLAEVG
KKFEKDTGIKVTVEHPDKLEEKFPQVAATGDGPDIIFWAHDRFGGYAQSG
LLAEITPDKAFQDKLYPFTWDAVRYNGKLIAYPIAVEALSLIYNKDLLPN
PPKTWEEIPALDKELKAKGKSALMFNLQEPYFTWPLIAADGGYAFKYENG
KYDIKDVGVDNAGAKAGLTFLVDLIKNKHMNADTDYSIAEAAFNKGETAM
TINGPWAWSNIDTSKVNYGVTVLPTFKGQPSKPFVGVLSAGINAASPNKE
LAKEFLENYLLTDEGLEAVNKDKPLGAVALKSYEEELAKDPRIAATMENA
QKGEIMPNIPQMSAFWYAVRTAVINAASGRQTVDEALKDAQTDDDD
KVPEFGSSRVDLQASL
SEQ ID No. 4
AKFLNGENGQPSKDQDIT
SEQ ID No. 5
DLKQNDTGTTQNQPD
SEQ ID No. 6
KYENGAGNGSWKVDGE
SEQ ID No. 7
TDKERQNGGQPPTTKDVT
SEQ ID No. 8
(Whole Ad36 fiber protein sequence
(0-371 aa)):
MSKRLRVEDDFNPVYPYGYARNQNIPFLTPPFVSSDGFQNFPPGVLSLKL
ADPIAIANGNVSLKVGGGLTVEQQSGKLSVDTKAPLQVANDNKLELSYDD
PFKVENNKLGIKAGHGLAVVTKENTSLPSLVGTLVVLTGKGIGTGSSAHG
GTIDVRLGEGGGLSFDEKGDLVAWDKKNDTRTLWTTPDPSPNCKVETARD
SKLTLALTKCGSQILATVSLLVVTGKYAIISDTVNPKQFSIKLLFNDKGV
LLSDSNLDGTYWNYRSNNNNIGTPYKEAVGFMPSTTAYPKPTNNTSTDPD
KKVSQGKNKIVSNIYLGGEVYQPGFIVVKFNQETDANCAYSITFDFGWGK
VYKDPIPYDTSSFTFSYIAQE
SEQ ID No. 9:
Whole Ad36 hexon protein sequence
(944 aa):
MATPSMMPQWAYMHIAGQDASEYLSPGLVQFARATDTYFSLGNKFRNPTV
APTHDVTTDRSQRLTLRFVPVDREDTTYSYKARFTLAVGDNRVLDMASTY
FDIRGVLDRGPSFKPYSGTAYNSLAPKGAPNSSQWTDKERQNGGQPPTTK
DVTKTFGVAARGGLHITDKGLQIGEDENNEDGEEEIYADKTFQPEPQVGE
ENWQDTDVFYGGRALKKETKMKPCYGSFARPTNEKGGQAKFLNGENGQPS
KDQDITLAFFDLKQNDTGTTQNQPDVVMYTENVYLETPDTHVVYKPGKED
TSSAANLTQQSMPNRPNYIGFRDNFVGLMYYNSTGNMGVLAGQASQLNAV
VDLQDRNTELSYQLLLDSLGDRTRYFSMWNSTVDSYDPDVRIIENHGVED
ELPNYCFPLDGSGSNTAYQGVKYENGAGNGSWKVDGEVASQNQIAKGNLY
AMEINLQANLWKSFLYSNVALYLPDSYKYTPANITLPTNTNTYEYMNGRV
VAPSLVDAYVNIGARWSLDPMDNVNPFNHHRNAGLRYRSMLLGNGRYVPF
HIQVPQKFFAIKNLLLLPGSYTYEWNFRKDVNMILQSSLGNDLRVDGASV
RFDSVNLYATLFPMARNTASTLEAMLRNDTNDQSFNDYLSAANMLYPIPA
KATNVPISIPSRNWAAFRGWSFTRLKTKETPSLGSGFDPYFVYSGSIPYL
DGTFYLNHTFKKVSIMFDSSVSWPGNDRLLTPNEFEIKRSVDGEGYNVAQ
CNMTKDWFLVQMLSHYNIGYQGFYVPEGYKDRMYSFFRNFQPMSRQVVDE
INYKDYKAVTLPFQHNNSGFTGYLAPTMRQGQPYPANFPYPLIGQTAVPS
VTQKKFLCDRVMWRIPFSSNFMSMGALTDLGQNMLYANSAHALDMTFEVD
PMDEPTLLYLLFEVFDVVRVHQPHRGVIEAVYLRTPFSAGNATT
SEQ ID No. 10:
Coating protein sequence for double
antigen ELISA (71-371 aa):
VEQQSGKLSVDTKAPLQVANDNKLELSYDDPFKVENNKLGIKAGHGLAVV
TKENTSLPSLVGTLVVLTGKGIGTGSSAHGGTIDVRLGEGGGLSFDEKGD
LVAWDKKNDTRTLWTTPDPSPNCKVETARDSKLTLALTKCGSQILATVSL
LVVTGKYAIISDTVNPKQFSIKLLFNDKGVLLSDSNLDGTYWNYRSNNNN
IGTPYKEAVGFMPSTTAYPKPTNNTSTDPDKKVSQGKNKIVSNIYLGGEV
YQPGFIVVKFNQETDANCAYSITFDFGWGKVYKDPIPYDTSSFTFSYIAQ
E

What is claimed is:

1. A method of detecting lipogenic adenovirus infection in a sample, said method comprising the steps of:
(a) providing a solid support coated with a first antibody capture polypeptide comprising at least 100 amino acids of an adenovirus type-36 (Ad-36) fiber coat protein, wherein the polypeptide does not include SEQ ID NO: 1;
(b) contacting the sample to the antibody capture polypeptide such that anti-antibody capture polypeptide antibodies, if present in the sample, bind to the antibody capture polypeptide to form an antibody capture polypeptide-anti-antibody capture polypeptide antibody complex;
(c) contacting the complex, if present, with an antibody detecting polypeptide comprising the at least 100 amino acids of the Ad-36 fiber coat protein; and
(d) detecting the presence, absence, or quantity of specific binding of the detection polypeptide to the complex, thereby detecting a lipogenic adenovirus infection in the sample.

2. The method of claim 1, further comprising the step of applying the first antibody capture polypeptide comprising at least 100 amino acids of an adenovirus type-36 (Ad-36) fiber coat protein, wherein the polypeptide does not include SEQ ID NO: 1 to the solid support for a period of time to allow the first antibody capture polypeptide to adhere to a surface of the solid support.

3. The method of claim 1, wherein the antibody capture polypeptide and/or the antibody detecting polypeptide comprises or consists of SEQ ID NO:10.

4. The method of claim 1, wherein the antibody capture polypeptide comprises a non-adenovirus heterologous amino acid sequence and step (c) further comprises contacting the sample with a competitor polypeptide comprising the heterologous amino acid sequence such that anti-competitor polypeptide antibodies, if present in the sample, bind to the competitor polypeptide.

5. The method of claim 4, wherein the competitor protein is a maltose binding protein.

6. The method of claim 1, wherein the solid support is linked to one or more further antibody capture polypeptide that comprises a fragment of an adenovirus hexon protein.

7. The method of claim 6, wherein the fragment of an adenovirus hexon protein comprise or consists of an amino acid sequence selected from the group consisting of SEQ ID NO: 4, SEQ ID NO: 5, SEQ ID NO: 6, and SEQ ID NO: 7.

8. The method of claim 1, wherein the solid support is linked to one or more further antibody capture polypeptide that consists of a peptide selected from SEQ ID NO: 4, SEQ ID NO: 5, SEQ ID NO: 6, and SEQ ID NO: 7.

9. The method of claim 1, wherein the antibody capture polypeptide comprises about 100 to about 400 amino acids of an Ad-36 fiber coat protein.

10. The method of claim 9, wherein the Ad-36 fiber coat protein is SEQ ID No. 8.

11. The method of claim 1, wherein the antibody detecting polypeptide is conjugated to a label that is capable of producing a measurable signal.

12. The method of claim 11, wherein the label is selected from the group consisting of horseradish peroxidase (HRP), I¹²⁵, alkaline phosphatase, fluorescein isothiocyanate (FITC), tetramethyl rhodamine isothiocyanate (TRITC), green fluorescent protein (GFP), allophycocyanin, phycocyanin, phycoerythrin, and phycoerythrocyanin.

13. The method of claim 1, wherein the sample is selected from the group consisting of biological sample, blood, semen, saliva, tears, nasal secretions, serum, cerebral fluid, urine, milk, and plasma.

14. An assay kit for detecting lipogenic adenovirus, said kit comprising:
a first antibody capture polypeptide comprising at least 100 amino acids of an adenovirus type-36 (Ad-36) fiber coat protein, wherein the polypeptide does not include SEQ ID NO: 1; and
an antibody detecting polypeptide comprising the at least 100 amino acids of the Ad-36 fiber coat protein.

15. The kit claim 14, wherein the antibody capture polypeptide and/or the antibody detecting polypeptide comprises or consists of SEQ ID NO:10.

16. The kit claim 14, wherein the first antibody capture polypeptide is linked to a solid support.

17. The kit claim 14, wherein the antibody capture polypeptide comprises about 100 to about 400 amino acids of an Ad-36 fiber coat protein.

18. The kit of claim 17, wherein the Ad-36 fiber coat protein is SEQ ID NO. 8.

19. The kit of claim 14, wherein the antibody capture polypeptide comprises a non-adenovirus heterologous amino acid sequence and the kit further comprises a competitor polypeptide comprising the heterologous amino acid sequence.

20. The kit of claim 19, wherein the competitor polypeptide is a maltose binding protein.

21. The kit of claim 14, further comprising, linked to the solid support, one or more further antibody capture polypeptide that comprises a fragment of an adenovirus hexon protein.

22. The kit of claim 21, wherein the fragment of an adenovirus hexon protein comprise or consists of an amino acid sequence selected from the group consisting of SEQ ID NO: 4, SEQ ID NO: 5, SEQ ID NO: 6, and SEQ ID NO: 7.

23. The kit of claim 14, further comprising a second antibody capture polypeptide comprises or consists of an amino acid sequence selected from SEQ ID NO:4 SEQ ID NO:5, SEQ ID NO:6, and SEQ ID NO:7.

24. The kit of claim 14, further comprises a label substrate, a wash solution, a blocking solution, a stopping solution, and a solid support.

25. The kit of claim 14, wherein said antibody detecting polypeptide is conjugated to a label.

26. The kit of claim 25, wherein the label is selected from the group consisting of horseradish peroxidase (HRP), I¹²⁵, alkaline phosphatase, fluorescein isothiocyanate (FITC), tetramethyl rhodamine isothiocyanate (TRITC), green fluorescent protein (GFP), allophycocyanin, phycocyanin, phycoerythrin, and phycoerythrocyanin.

27. An assay device used for detecting lipogenic adenovirus infection, said device comprising:
a solid support linked to a first antibody capture polypeptide comprising at least 100 amino acids of an adenovirus type-36 (Ad-36) fiber coat protein, wherein the polypeptide does not include SEQ ID NO: 1.

28. The assay device of claim 27, wherein the antibody capture polypeptide comprises or consists of SEQ ID NO:10.

29. The assay device of claim 27, wherein said solid support is coated with a second antibody capture polypeptide that comprises a fragment of an adenovirus hexon protein.

30. The assay device of claim 29, wherein the second antibody capture polypeptide comprises or consists of a polypeptide selected from SEQ ID NO: 2, SEQ ID NO: 4, SEQ ID NO: 5, SEQ ID NO: 6, and SEQ ID NO: 7.

31. The assay device of claim 27, wherein the antibody capture polypeptide comprises about 100 to about 400 amino acids of an Ad-36 fiber coat protein.

32. The assay device of claim 31, wherein the Ad-36 fiber coat protein is SEQ ID NO. 8.

33. The assay device of claim 27, wherein said solid support may be selected from a microtiter plate, a bead, a rod, nitrocellulose, a dipstick, or petri dish.

34. The assay device of claim 27, further comprising an anti-antibody capture polypeptide linked to the first antibody capture polypeptide forming an anti-antibody capture polypeptide—first antibody capture polypeptide complex.

35. The assay device of claim 34, further comprising an antibody detecting polypeptide linked to the complex.

36. The assay device 35, wherein the antibody detecting polypeptide is conjugated to a label.


SEQUENCE LISTING (15 sequences)

SEQ ID NO: 1 - protein, 70 aa, Artificial Sequence
  synthetic adenovirus type-36 (Ad-36) fiber coat protein polypeptide, adenovirus type-36 fiber coat protein amino acids 1-70
MSKRLRVEDDFNPVYPYGYARNQNIPFLTPPFVSSDGFQNFPPGVLSLKLADPIAIANGNVSLKVGGGLT

SEQ ID NO: 2 - protein, 23 aa, Artificial Sequence
  synthetic adenovirus type-36 (Ad-36) hexon protein polypeptide, adenovirus type-36 hexon protein amino acids 168-190, second antibody capture polypeptide, potential optimizer peptide
DKGLQIGEDENNEDGEEEIYADK

SEQ ID NO: 3 - protein, 412 aa, Escherichia coli
  maltose binding protein (MBP)
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTDDDDKVPEFGSSRVDLQASL

SEQ ID NO: 4 - protein, 18 aa, Artificial Sequence
  synthetic adenovirus type-36 (Ad-36) hexon protein polypeptide, adenovirus type-36 hexon protein amino acids 239-256, second antibody capture polypeptide, potential optimizer peptide
AKFLNGENGQPSKDQDIT

SEQ ID NO: 5 - protein, 15 aa, Artificial Sequence
  synthetic adenovirus type-36 (Ad-36) hexon protein polypeptide, adenovirus type-36 hexon protein amino acids 261-275, second antibody capture polypeptide, potential optimizer peptide
DLKQNDTGTTQNQPD

SEQ ID NO: 6 - protein, 16 aa, Artificial Sequence
  synthetic adenovirus type-36 (Ad-36) hexon protein polypeptide, adenovirus type-36 hexon protein amino acids 422-437, second antibody capture polypeptide, potential optimizer peptide
KYENGAGNGSWKVDGE

SEQ ID NO: 7 - protein, 18 aa, Artificial Sequence
  synthetic adenovirus type-36 (Ad-36) hexon protein polypeptide, adenovirus type-36 hexon protein amino acids 134-153, second antibody capture polypeptide, potential optimizer peptide
TDKERQNGGQPPTTKDVT

SEQ ID NO: 8 - protein, 371 aa, Adenovirus type 36
  adenovirus type-36 (Ad-36) fiber coat protein, antibody detection polypeptide
MSKRLRVEDDFNPVYPYGYARNQNIPFLTPPFVSSDGFQNFPPGVLSLKLADPIAIANGNVSLKVGGGLTVEQQSGKLSVDTKAPLQVANDNKLELSYDDPFKVENNKLGIKAGHGLAVVTKENTSLPSLVGTLVVLTGKGIGTGSSAHGGTIDVRLGEGGGLSFDEKGDLVAWDKKNDTRTLWTTPDPSPNCKVETARDSKLTLALTKCGSQILATVSLLVVTGKYAIISDTVNPKQFSIKLLFNDKGVLLSDSNLDGTYWNYRSNNNNIGTPYKEAVGFMPSTTAYPKPTNNTSTDPDKKVSQGKNKIVSNIYLGGEVYQPGFIVVKFNQETDANCAYSITFDFGWGKVYKDPIPYDTSSFTFSYIAQE

SEQ ID NO: 9 - protein, 944 aa, Adenovirus type 36
  adenovirus type-36 (Ad-36) hexon protein, second antibody capture polypeptide
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWTDKERQNGGQPPTTKDVTKTFGVAARGGLHITDKGLQIGEDENNEDGEEEIYADKTFQPEPQVGEENWQDTDVFYGGRALKKETKMKPCYGSFARPTNEKGGQAKFLNGENGQPSKDQDITLAFFDLKQNDTGTTQNQPDVVMYTENVYLETPDTHVVYKPGKEDTSSAANLTQQSMPNRPNYIGFRDNFVGLMYYNSTGNMGVLAGQASQLNAVVDLQDRNTELSYQLLLDSLGDRTRYFSMWNSTVDSYDPDVRIIENHGVEDELPNYCFPLDGSGSNTAYQGVKYENGAGNGSWKVDGEVASQNQIAKGNLYAMEINLQANLWKSFLYSNVALYLPDSYKYTPANITLPTNTNTYEYMNGRVVAPSLVDAYVNIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATLFPMARNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFYVPEGYKDRMYSFFRNFQPMSRQVVDEINYKDYKAVTLPFQHNNSGFTGYLAPTMRQGQPYPANFPYPLIGQTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATT

SEQ ID NO: 10 - protein, 301 aa, Artificial Sequence
  synthetic adenovirus type-36 (Ad-36) fiber coat protein polypeptide, adenovirus type-36 fiber coat protein amino acids 71-371, coating protein for double antigen ELISA, antibody capture polypeptide, antibody detecting polypeptide
VEQQSGKLSVDTKAPLQVANDNKLELSYDDPFKVENNKLGIKAGHGLAVVTKENTSLPSLVGTLVVLTGKGIGTGSSAHGGTIDVRLGEGGGLSFDEKGDLVAWDKKNDTRTLWTTPDPSPNCKVETARDSKLTLALTKCGSQILATVSLLVVTGKYAIISDTVNPKQFSIKLLFNDKGVLLSDSNLDGTYWNYRSNNNNIGTPYKEAVGFMPSTTAYPKPTNNTSTDPDKKVSQGKNKIVSNIYLGGEVYQPGFIVVKFNQETDANCAYSITFDFGWGKVYKDPIPYDTSSFTFSYIAQE

SEQ ID NO: 11 - protein, 12 aa, Artificial Sequence
  synthetic adenovirus type-36 (Ad-36) fiber coat protein polypeptide, adenovirus type-36 fiber coat protein amino acids 348-354, Ad36 specific peptide
WGKVYKDPIPYD

SEQ ID NO: 12 - protein, 10 aa, Artificial Sequence
  synthetic adenovirus type-36 (Ad-36) fiber coat protein polypeptide, adenovirus type-36 fiber coat protein amino acids 195-204, Ad36 specific peptide
VETARDSKLT

SEQ ID NO: 13 - protein, 7 aa, Artificial Sequence
  synthetic adenovirus type-36 (Ad-36) fiber coat protein polypeptide, adenovirus type-36 fiber coat protein amino acids 143-149, Ad36 specific peptide
GTGSSAH

SEQ ID NO: 14 - protein, 14 aa, Artificial Sequence
  synthetic adenovirus type-36 (Ad-36) fiber coat protein polypeptide, adenovirus type-36 fiber coat protein amino acids 300-329, Ad36 specific peptide
LGGEVYQPGFIVVK

SEQ ID NO: 15 - protein, 6 aa, Artificial Sequence
  synthetic His Tag composed of six histidines
HHHHHH